(19) United States
(12) Patent Application Publication
(10) Pub. No.: US 2005/0241009 A1
(43) Pub. Date: Oct. 27, 2005

(54) Development of a murine model of HIV-1 infection on the basis of construction of EcoHIV, a chimeric, molecular clone of human immunodeficiency virus type 1 and ecotropic moloney murine leukemia virus competent to infect murine cells and mice

(76) Inventors: Mary Jane Potash, Scarsdale, NY (US); David J. Volsky, Scarsdale, NY (US)

Correspondence Address: LESLIE GLADSTONE RESTAINO;BROWN RAYSMAN MILLSTEIN & STEINER LLP, 163 MADISON AVENUE, PO BOX 1989, MORRISTOWN, NJ, 07962-1989, US

(21) Appl. No.: 11/041,158

(22) Filed: Jan. 20, 2005

Related U.S. Application Data
(60) Provisional application No. 60/564,505, filed on Apr. 21, 2004.

(51) Int. Cl. A01K 67/027; C12N 15/867

(52) U.S. Cl. 800/10; 800/14; 800/18; 435/456

(57) ABSTRACT

The present invention provides a chimeric HIV-1 construct, EcoHIV, capable of replication in a rodent cell. The invention also provides a convenient and safe rodent model of HIV-1 infection and AIDS. Methods for producing a rodent model of HIV-1 infection are also provided. Additionally, the invention provides the means to test immunogenic compositions or pharmaceutical interventions effective in preventing infection, reducing viral load, or reducing disease symptoms in a subject.

### CROSS REFERENCE TO RELATED APPLICATIONS

The present application claims the benefit of U.S. Application Ser. No. 60/564505, filed Apr. 21, 2004, which is incorporated herein by reference.

### STATEMENT OF GOVERNMENT INTEREST

This invention was made with government support under NIH Grant No. R21 DA-14934. As such, the United States government may have certain rights in this invention.

### BACKGROUND OF THE INVENTION

AIDS—acquired immunodeficiency syndrome—was first reported in the United States in 1981 and has since become a major worldwide epidemic. AIDS is caused by the human immunodeficiency virus (HIV). By killing or damaging cells of the body's immune system, HIV progressively destroys the body's ability to fight infections and certain cancers. There are two types of HIV, HIV-1 and HIV-2. HIV-1 is the more virulent type, and naturally infects human beings, as well as a small number of non-human primates. Compared with persons infected with HIV-1, those with HIV-2 are less infectious early in the course of infection. As the disease advances, HIV-2 infectiousness seems to increase. However, compared with HIV-1, the duration of this increased infectiousness is shorter. There is currently no cure for HIV infection or AIDS.

Studies of HIV-1 pathogenesis have been hampered because of lack of a suitable animal model. Because the mouse immune system has been extensively researched, a murine model of HIV-1 infection would be ideal and extremely valuable for evaluation of therapies and vaccines. However, prior to the present invention mouse cells were believed to be effectively resistant to HIV-1 infection, replication and spread. Multiple blocks to productive infection of mouse cell lines have been reported (Levy, et al., AIDS retrovirus (ARV-2) clone replicates in transfected human and animal fibroblasts, Science 232:998-1001 (1986); Maddon, et al., The T4 gene encodes the AIDS virus receptor and is expressed in the immune system and the brain, Cell 47:333-348 (1986); Winslow, et al., The blocks to human immunodeficiency virus type 1 Tat and Rev functions in mouse cell lines are independent, J. Virol. 67:2349-2354 (1993)). For example, blocks to HIV-replication have been reported at the level of virus entry, transcription, RNA transport, protein processing, and viron assembly (Bieniasz, et al., Multiple blocks to human immunodeficiency virus type 1 replication in rodent cells. J. Virol. 74:9868-9877 (2000); Mariani, et al., A block to human immunodeficiency virus type 1 assembly in murine cells, J. Virol. 74:3859-3870 (2000)). However, the inventors recently published findings indicating the primary murine cells are susceptible to HIV-1 replication in tissue culture (Nitkiewicz et al., Productive infection of primary murine astrocytes, lymphocytes, and macrophages by human immunodeficiency virus type 1 in culture. J Neurovirol. 10:400-408 2004).

Since primary mouse cells are permissive to HIV-1 expression, the principal difficulty in constructing mouse models of HIV-1 infection rests in achieving efficient virus binding and entry into murine cells. Previous models employing mice or rats transgenic for the human receptors for HIV-1, CD4 and either CCR5 or CXCR4, failed to exhibit significant susceptibility to HIV-1 infection (Sawada et al., J. Exp. Med, 187, 1439-1449, 1998; Browning et al., Proc. Natl. Acad. Sci. USA, 94, 14637-14641, 1997). The inventors disclose herein, a different strategy based upon studies of infection in culture by HIV-1 enveloped by heterologous proteins (Nitkiewicz et al., Journal of Neuro Virology, 10:400-408, 2004; Hinkula et al., Cells Tissues Organs, 177, 169-184, 2004; Page et al., J. Virol., 64, 5270-5276, 1990). Rather than endow mice with receptors for HIV-1, the inventors constructed HIV-1 species with receptors for mouse cells. As described in the examples, which follow, the inventors converted the host species range of HIV-1 from primate to rodent by replacing the coding region of its surface envelope glycoprotein, gp120, with the envelope-coding region from ecotropic MLV that restricts the replication of the virus to rodents (Albritton et al., Cell, 57, 659-666, 1989). Two such chimeric viruses were constructed, EcoHIV on a backbone of Clade B NL4-3 (Adachi et al., J. Virol., 59, 284-291, 1986) and EcoNDK on a backbone of Clade D NDK (Ellrodt et al., Lancet, 1, 1383-1385, 1984). The chimeric virus replicated in murine lymphocytes but not human lymphocytes in culture. EcoHIV and EcoNDK established systemic infection in mice after one inoculation. Importantly, this experimental infection reproduced several major characteristics of HIV-1 infection of human beings including virus targeting to lymphocytes and macrophages, induction of immune responses to viral proteins, neuroinvasiveness, and elevation of expression of inflammatory and antiviral factors in the brain. These findings indicate that EcoHIV and similar chimeric viruses can serve as important tools for investigation of HIV-1 disease and intervention in a versatile and convenient animal host.

### SUMMARY OF THE INVENTION

The present invention is directed to a process for constructing and producing an HIV-1 construct capable of infecting rodent cells. This allows for the development of a convenient and safe mouse model of HIV-1 infection which can be used for: 1) testing potential routes to HIV-1 pathogenesis in an animal that is susceptible to HIV-1 infection and spread; 2) testing potential antiviral therapies for HIV-1; and 3) testing potential HIV-1 vaccines in an immunocompetent host which is susceptible to HIV-1 infection. The infectious HIV-1 of the invention, EcoHIV, is a molecular virus chimera which was constructed based upon the full length infectious HIV-1 molecular clone, NL4-3, and the full length infectious ecotropic MuLV clone, NCAC. A similar virus chimera was also constructed based upon the full length infectious molecular clone of Clade D HIV-1, NDK. EcoHIV and EcoNDK can be recovered from harvest of the culture medium from mammalian cells transfected with the EcoHIV plasmid. The virus is competent to replicate in primary mouse splenic lymphocytes, producing HIV-1 RNA, HIV-1 core antigen p24, and fusogenic viral envelope. EcoHIV was shown to infect conventional immunocompetent mice after intravenous inoculation by detection of viral DNA in spleen, macrophages, and brain cells and by detection of serum antibodies to viral Tat and Gag proteins. EcoNDK was also shown to infect conventional mice.

In contrast with other strategies used to create animal models of HIV infection, EcoHIV replaces HIV-1 envelope glycoprotein with MuLV envelope which provides the virus with receptors competent to interact directly with mouse cells. Other approaches instead modify the mouse itself by grafting human tissue or by introduction of human genes. Additionally, EcoHIV is particularly advantageous because it is designed to be non-infectious to humans and it was shown not to infect human peripheral blood lymphocytes in culture. Thus, from a researcher's perspective, EcoHIV provides a less hazardous alternative to using HIV-1 or its derivatives.

Accordingly, the present invention provides compositions and methods for use in developing a rodent model of HIV-1 infection and AIDS. More specifically, the invention provides compositions and methods for use in developing a murine model of HIV-1 infection and AIDS for investigation of viral replication, control and pathogenesis.

The invention provides a chimeric HIV construct capable of infecting a rodent cell, comprising coding and regulatory regions of the HIV-1 genome, and a heterologous viral envelope. In a specific embodiment, the present invention provides a chimeric HIV-1 construct capable of infecting a rodent cell, comprising coding and regulatory regions of the HIV-1 genome, wherein the complete or partial coding region of gp120 is replaced by the coding region for a heterologous viral envelope. Importantly, a molecular clone of HIV-1 of any clade or construction can be used for construction of the chimeric construct of the invention. Similarly, a molecular clone of any heterologous viral envelope that permits infection of rodent cells can be used for construction of the chimeric construct of the present invention. In a preferred embodiment, the complete or partial coding region of gp120 is replaced by the coding region for ecotropic murine leukemia virus gp80.

The present invention also provides a method for producing a rodent model of HIV-1 infection comprising administering the EcoHIV construct of the invention to a rodent. In another embodiment, the invention provides a rodent model of HIV-1 infection and propagation in the rodent host in which at least the somatic cells are susceptible to infection by the EcoHIV construct of the present invention and wherein expression of the construct is sufficient to effect phenotypic changes consistent with HIV-1 pathology. In a preferred embodiment, the rodent host is a mouse. However, any rodent may be used as a model in the present invention including, but not necessarily limited to, all species of mouse and rat.

The present invention additionally provides a rodent model of AIDS in which at least the somatic cells are susceptible to infection by the EcoHIV construct and wherein expression of the construct is sufficient to effect phenotypic changes consistent with AIDS pathology. In yet another embodiment, the invention provides a model of HIV-1 infection of an immunocompetent rodent suitable for testing HIV-directed immunogenic compositions or vaccines, or other pharmaceutically or veterinarilly suitable compositions for their efficacy in preventing infection in a subject inoculated with the EcoHIV construct.

The present invention also provides a model of HIV-1 infection of an immunocompetent rodent suitable for testing HIV-1 directed immunogenic compositions or vaccines, or other pharmaceutically or veterinarilly suitable compositions for their efficacy in reducing viral load in a subject inoculated with the EcoHIV construct. The invention further provides a model of HIV-1 infection of an immunocompetent rodent suitable for testing HIV-1 directed immunogenic compositions or vaccines, or other pharmaceutically or veterinarilly suitable compositions for their efficacy in ameliorating disease in a subject inoculated with the EcoHIV construct. In addition, the present invention provides a rodent model for treatment of HIV-1 infection in which somatic cells are susceptible to infection by the EcoHIV construct and pharmaceutical interventions may be tested for their efficacy in reducing viral load.

Additional aspects of the present invention will be apparent in view of the description which follows.

### DESCRIPTION OF THE FIGURES

FIG. 1 depicts a map of the construction of the chimeric HIV-1, EcoHIV. The virus carries all HIV-1 structural and regulatory genes, named above or under bars, except most of the coding region of gp120. 1405 bp of gp120 was excised and replaced by the coding region of the MLV ecotropic envelope gene gp80 with its stop codon in place. HIV-1 cis-regulatory elements were preserved and expression of the entire construct is driven by the HIV-1 LTR. (B) Cultures were sampled over time for p24 expression by immunoblot, compared to human CEM cells infected by HIV-1 or EcoHIV. The amount of p24 detected by Elisa in EcoHIV-infected lymphocyte cultures underestimates the protein detected by Western blot. (C-D) At seven days after infection cells were (C) stained for HIV-1 antigens, right panel, uninfected cells were stained in parallel, left panel; or (D) co-cultured with a nine-fold excess of uninfected cells and examined for syncytia after two days, right panel, uninfected cells co-cultured in parallel, left panel.

FIG. 2 shows detection of HIV-1 DNA in various compartments of EcoHIV infected mice six weeks after infection (Panel A) Spleen and brain DNA were standardized by β-globin content for quantitation using standard curves as shown; macrophage samples are not standardized because of low amount of DNA obtained. 1-5: infected mice, C: uninfected mouse. Panel B shows viral DNA in CD4-positive but not CD4-negative splenic lymphocytes. 18: infected mouse, C: uninfected mouse. Panel C shows a dose response to EcoHIV infection testing viral DNA in the spleen. 26-29 and 34-37: infected mice, C: uninfected mouse. The table summarizes detection of EcoHIV DNA in various tissues in six independent experimental infections.

FIG. 3 demonstrates that EcoHIV infected mice show increased expression of MCP-1 and complement C3 RNA in the brain. Gene expression was probed by RT-PCR on RNA isolated from brains of mice #6-10 and control animal using mouse-specific primers; downstream primer was used for first-strand cDNA synthesis; the number of DNA PCR cycles is indicated. HIV-1 infected human fetal astrocytes (V1, V2) probed with human C3 primers 7 days p.i. served as positive control for C3. C1, C2; uninfected astrocytes. RPS9: RT-PCR of mouse ribosomal small protein 9 RNA, used for standardization of samples. The figure shows ethidium bromide staining.

FIG. 4 shows detection of expressed HIV-1 genome in vivo and reactivation of virus in culture. Isolated spleen cells from mice 6 and 8 were either directly tested for the presence of Vif RNA by RT-PCR (Ex vivo) or cultured first for 2 days in the presence of concanavalin A to activate cells and virus and then tested (In vitro). C: uninfected mouse cells; CEMxHIVHIV-infected CEM cells.

FIG. 5 shows rescue of EcoHIV from spleens of infected animals by serial cocultivation with uninfected mouse spleen cells. The culture was harvested at the fourth passage, stained with serum from an HIV-1 infected person and FITC-labeled anti-human IgG to detect HIV-1 antigens.

FIG. 6 demonstrates that EcoHIV infected mice produce anti-HIV-1 antibodies. A-B: Sera were collected 12 weeks after EcoHIV infection (6 weeks after infection for mouse #15), diluted as indicated in the Figure, and tested for binding HIV-1 proteins in solid phase. Antibody binding was detected using radioiodinated anti-mouse Ig. (A) Recombinant HIV-1 p55 was bound to wells at 250 ng per well. (B) Recombinant HIV-1 Tat was bound to wells at 500 ng per well. Both recombinant proteins were provided by the NIH Aids Research Reagent Program.

FIG. 7 shows neuropathological findings in brains of mice 6-12 weeks after EcoHIV infection. (A-C), cellular aggregate in the region inferior to basal ganglia from mouse #129-8 as seen at low, medium, and high power (H&E). Note increased vascularity, pyknotic cells, and the possible multinucleated giant cell in the center of the field. (D) region inferior to basal ganglia in control uninfected mouse #129-C. No lesions were found. Medium power (H&E). (E), another, similar aggregate near to that shown in (A-C), inferior to basal ganglia in mouse #129-8, at medium power (H&E). (F), Leptomeningeal infiltrate, with mononuclear cells, mouse #129-1, medium power (H&E). Mouse #129-1 and mouse #129-8 were evaluated 6 and 12 weeks after EcoHIV inoculation, respectively.

FIG. 8 shows impaired immune activation in lymphocytes from mice infected by EcoHIV. Spleen cells were harvested from mice infected by two injections of EcoHIV or from uninfected mice and were activated to the expression of the cytokine interferon-gamma by culture with concanavalin A. Interferon-gamma production was detected by flow cytometry. Infected mouse 2′-1 shows profound impairment of immune activation and infected mouse 2′-2 shows some reduction relative to the uninfected mouse.

FIG. 9 shows the infectivity and cellular response of EcoNDK, a chimeric virus based upon Clade D HIV-1 NDK. Panel A shows EcoNDK viral DNA in spleen and brain three weeks after inoculation; a standard curve of amplification of plasmid DNA is at the right. Panel B shows quantitative RT-PCR of total cellular RNA from brain tissue of infected mice NDK 8 and 9, comparing levels of transcripts to levels found in the control brain. Asterisks indicate differences compared to control at p<0.05 by t test. Panel C shows immunocytochemical staining for STAT-1 in mouse cortical brain sections, arrows indicate examples of more intense staining for STAT-1 in infected mouse NDK 8 (right panel) compared to the control brain (left panel). The final magnification as shown is 360×.

FIG. 10 (A-B) depicts a scheme for insertion of gp120 regions into EcoHIV.

### DETAILED DESCRIPTION OF THE INVENTION

The versatility in inbred and genetically engineered mouse strains, combined with the extensive knowledge of the murine immune system makes the mouse an ideal animal for HIV and AIDS research. The present invention establishes for the first time a useful mouse model of HIV-1 infection and AIDS which possesses many advantages over prior animal models of HIV and AIDS. A specific viral construct, EcoHIV, is provided which is capable of infecting rodent cells. The inventors have found that EcoHIV is infectious to normal mouse lymphocytes, producing infectious progeny virus. The virus of the present invention has the particular advantage of being non-infectious to human cells, making it safe for researchers compared with HIV-1 or its derivatives, SHIV. The inventors disclose herein that EcoHIV infects conventional immunocompetent mice and induces antiviral immune responses. EcoHIV infected mice can be used for studies of viral replication, antiviral therapies, vaccines, and pathogenesis.

Accordingly, the invention provides a chimeric HIV construct capable of infecting a rodent cell, comprising coding and regulatory regions of the HIV-1 genome, and a heterologous viral envelope. In a specific embodiment, the present invention provides a chimeric viral construct capable of infecting a rodent cell comprising coding and regulatory regions of the HIV-1 genome, wherein the complete or partial coding region of gp120 is replaced by the coding region for a heterologous viral envelope. Importantly, a molecular clone of HIV-1 of any clade or construction can be used for construction of the chimeric construct of the invention. A molecular clone of any heterologous viral envelope that permits infection of rodent cells can be used for construction of the chimeric construct of this invention. In a preferred embodiment, the complete or partial coding region of gp120 is replaced by the coding region for ecotropic murine leukemia virus gp80.

The present invention also provides a method for producing a rodent model of HIV-1 comprising administering the EcoHIV construct of the invention to a rodent. In one embodiment of the invention, infectious EcoHIV is recovered by transfection of an EcoHIV bacterial plasmid into a mammalian cell line in culture, and harvesting the culture medium from the transfected cells. Many mammalian cell lines can be used for transfection. In an embodiment of the invention, human embryonic kidney cell line 293T is used. The recovered virus is competent to replicate in primary mouse splenic lymphocytes in culture, producing HIV-1 RNA, HIV-1 core antigen p24 and fusogenic viral envelope.

In another embodiment of the invention, replication competent EcoHIV is produced by rodent cells infected in tissue culture. In an embodiment of the present invention, the rodent can be infected with the replication competent EcoHIV by intraperitoneal or intravenous injection. In another embodiment of the present invention, the rodent can be infected with isogenic rodent cells expressing the replication competent EcoHIV. Any rodent may be used as a model in the present invention including, but not necessarily limited to, all species of mouse and rat. In a preferred embodiment, a mouse is used as the rodent model.

In a further embodiment, the invention provides a rodent model of HIV-1 infection and propagation in a rodent host in which at least the somatic cells are susceptible to infection by the EcoHIV construct of the present invention and wherein expression of the construct is sufficient to effect phenotypic changes consistent with HIV-1 pathology. The term “propagation in a rodent host” as used in the present invention refers to the capability of the infectious viral construct to go through more than one cycle of replication in rodent cells and rodents. The skilled artisan can readily identify the occurrence of clinical symptoms consistent with HIV-1 infection and pathology. In a preferred embodiment, the rodent model is a mouse. However, any rodent may be used as a model in the present invention, including but not necessarily limited to, all species of mouse and rat.

The present invention additionally provides a rodent model of AIDS in which at least the somatic cells are susceptible to infection by the EcoHIV construct and wherein expression of the construct is sufficient to effect phenotypic changes consistent with AIDS pathology. The skilled artisan can readily identify the occurrence of clinical symptoms consistent with AIDS pathology.

In yet another embodiment, the invention provides a model of HIV-1 infection of an immunocompetent rodent suitable for testing an HIV-1 directed immunogenic composition or vaccine for its efficacy in preventing infection or reducing viral load in a subject inoculated with the EcoHIV construct. Additionally, the invention provides a model of HIV-1 infection of an immunocompetent rodent suitable for testing an HIV-1 directed immunogenic composition or vaccine for its efficacy in ameliorating disease symptoms in a subject inoculated with the EcoHIV construct. As used in the present invention, the term “immunogenic composition” refers to a composition comprising an antigenic molecule where administration of the composition to a subject results in the development in the subject of a humoral and/or cellular immune response to the antigenic molecule or cross reacting molecules. As used in the present invention, the term ameliorating disease refers to reducing HIV-1 infection associated symptoms or pathology or AIDS associated symptoms or pathology. As used in the present invention, the term preventing disease refers to preventing the initiation of HIV-1 infection or AIDS, preventing HIV-1 infection or AIDS, delaying the initiation of HIV-1 infection or AIDS, preventing the progression or advancement of HIV-1 infection or AIDS, slowing the progression or advancement of HIV-1 infection or AIDS, and delaying the progression or advancement of HIV-1 infection or AIDS.

The present invention also provides a rodent model for treatment of HIV-1 infection in which somatic cells are susceptible to infection by the EcoHIV construct and pharmaceutically acceptable compounds or veterinarilly acceptable compounds can be tested for their efficacy in reducing viral load. As used herein, the terms “pharmaceutically acceptable” or “veterinarilly acceptable” refer to material that may be administered to a subject in a composition without causing any deleterious or otherwise undesirable biological effects.

A model of AIDS treatment is also provided, wherein expression of the construct is sufficient to effect phenotypic changes consistent with AIDS pathology that can be tested for amelioration by pharmaceutically acceptable or veterinarilly acceptable compounds.

The following examples illustrate the present invention, and are set forth to aid in the understanding of the invention, and should not be construed to limit in any way the scope of the invention as defined in the claims which follow thereafter.

### EXAMPLES

### Example 1

Materials and Methods

### Design of EcoHIV, a Chimeric HIV-1 Construct that Targets Rodent Cells

EcoHIV is designed to carry all the coding and regulatory regions of the HIV-1 genome, except for the gene encoding the viral envelope glycoprotein, gp120 that targets the virus to the CD4 bearing cells. In its place, the coding region for ecotropic murine leukemia virus gp80, that targets the virus to rodent, but not to human cells, was inserted. Specifically, EcoHIV was constructed based upon the full-length infectious HIV-1 molecular clone, NL4-3, and the full-length infectious ecotropic MuLV clone, NCAC. Referring to the numbering systems of NL4-3 and NCAC, respectively, a fragment from nucleotide 6310 to 7750, encoding amino acids 31 to 510 of HIV-1 gp120 was excised from NL4-3 and replaced in frame with a fragment from nucleotide 6129 to 8823 of NCAC, encoding amino acids 2 to 697 and the termination codon of gp80. The resulting envelope glycoprotein contains 727 amino acids. The overlapping coding sequences of Vpu, Tat, and Rev as well as the cis Rev response elements of HIV-1 are preserved in the final construct. The resulting EcoHIV resides on the bacterial plasmid, pUC18. Infectious EcoHIV can be recovered by transfection of the bacterial plasmid into a mammalian cell line in culture, for example the human embryonic kidney cell line, 293T, and harvest of the culture medium from transfected cells. The culture medium contains a virus competent to replicate in primary mouse splenic lymphocytes, producing HIV DNA, HIV-1 RNA, HIV-1 core antigen p24 and fusogenic viral envelope. Viral infection can be maintained by the addition of uninfected splenic lymphocytes to infected cells, a method in which EcoHIV is produced in an infectious form and transmitted in culture.

EcoHIV is prepared for infection by transfection of plasmid DNA into human embryonic kidney cells in culture, bypassing receptor-mediated entry. In several tissue culture studies, it was confirmed that EcoHIV is replication competent in mouse lymphocytes but unable to infect either primary or transformed human cells. Infected mouse lymphocytes produced HIV-1 Gag protein and mature core antigen p24 as shown by Western blot and immunofluorescent staining.

EcoNDK was similarly constructed using Clade D NDK as backbone and MLV NCAC to provide the gp80 coding region. Nucleotides 5846-7265, encoding gp120 were deleted from NDK and nucleotide 6129 to 8823 of NCAC, encoding amino acids 2 to 697 and the termination codon of gp80 were inserted in frame. EcoNDK resides on the bacterial plasmid pUC18 and is prepared by transfection of plasmid DNA in culture.

### Mice, Cells and Sample Preparation

All animal studies were conducted with the approval of the St. Luke's-Roosevelt IACUC. Adult female BALB/c, 129P3, and 129X1 mice were purchased from Jackson Labs (Bar Harbor, Me.). For inoculation of EcoHIV, mice were anesthetized with isoflurane and a 0.1 ml solution of virus stock was injected into the tail vein. Prior to euthanasia, mice were anesthetized for bleeding, then they were subjected to carbon dioxide asphyxiation, spleens, brains, lungs, and kidneys were surgically removed, and peritoneal macrophages were harvested by peritoneal wash. Spleen cell suspensions were prepared and cultures were established as described (Nitkiewicz et al., Journal of Neuro Virology, 10:400-408, 2004). Cells were fractionated into CD4-bearing and CD4-negative using Dynabeads (Dynal, Oslo, Norway) and fractionation was confirmed by flow cytometry.

Splenic lymphocytes were infected in culture with 0.5 pg p24 EcoHIV per cell and harvested later for immunoblot or microscopy. Transformed human CEM cells infected with HIV-1 or EcoHIV served as controls. For PCR, brain, kidney, or lung tissue was weighed and then homogenized using a disposable pestle. DNA was isolated from cells or tissues using DNAzol (Invitrogen, Carlsbad, Calif.), precipitated with ethanol, and resuspended in water; RNA was isolated with either Trizol (Invitrogen) or with Rneasy (Qiagen, Valencia, Calif.).

### PCR

EcoHIV DNA present in 5×10⁵ lymphocytes or 1.25 mg brain, kidney, or lung was amplified using primers NE5 (5′-ATGATCTGTAGTGCTGCGCGTTCAACG-3′) and Eco3 (5′-GAGCCGGGCGAAGCAGTACTGACCCCTC-3′) that span the joint between HIV-1 and MLV, amplification was conducted as described (Chowdhury et al., J. Neuro Virol., 8, 599-610, 2002), and reaction products were detected using the 32P-labeled probe, EcoP3 (5′-GGTTAACCCGCGAGGCCCCCTAATCCCC-3′). The single copy cellular gene, β globin, was amplified in parallel to standardize DNA input. For detection of singly spliced Vif RNA, cDNA was prepared and amplified as described (Chowdhury et al., J. Neuro Virol., 8, 599-610, 2002) using sense primer nt 616-641, antisense primer nt 5071-5091, and radiolabeled probe nt 5127-5156 with numbering according to the NL4-3 genome.

### Quantitative Real-Time RT-PCR (QPCR)

For QPCR, RNA was isolated from brain tissue using a modified Trizol protocol optimized to handle the high lipid component of brain homogenates as described (http://www.fgc.urmc.rochester.edu). RNA quality was assessed in the Agilent 2100 bioanalyzer. Twenty ng of total RNA was used for Whole Transcriptome Amplification (WTA), based on Ribo-SPIAT technology from NuGEN Technologies Inc, to generate cDNA, according to the manufacturer's protocol (http://www.nugeninc.com). The size distribution and linearity of amplification was measured prior to quantitative analysis. Expression of selected cellular genes in the brain was examined using Taqman chemistry with MGB probes and primers selected from the Applied Biosystems Assay on Demand program. The relative efficiency of all assays was compared to glyceraldehyde 3 phosphate dehydrogenase and was within the parameters established for ΔΔ Ct analysis. Following probe and primer optimization all cDNA's were diluted 1:300 following amplification and used as described previously (Kim et al., J. Neuroimmunol., 157:17-26, 2004). Test transcript values were normalized to levels of glyceraldehyde 3 phosphate dehydrogenase and presented as fold change versus the levels in control brain samples.

### Fluorescence Microscopy

Microscopy was conducted as described (Nitkiewicz et al., Journal of Neuro Virology, 10:400-408, 2004) using serum from an AIDS patient and FITC-labeled anti-human Ig (Sigma, St. Louis, Mo.). Nuclei were stained with propidium iodide. Images were captured using a Zeiss Model Axioplan 2 microscope (Carl Zeiss) with a HAMAMATSU ORCA-ER digital carnera (HAMAMATSU Corp)

### Immunoblot and Radioimmunoassay

Immunoblot was conducted as described (Nitkiewicz et al., Journal of Neuro Virology, 10:400-408, 2004). Solid phase-radioimmunoassays (RIA) were constructed by coupling purified recombinant viral proteins (NIH AIDS Research Reagent Repository) to immunolon wells (Dynatech Laboratories, Chantilly, Va.) using 250 ng Gag per well or 500 ng Tat per well. Dilutions of mouse sera were added to wells and immunoglobulin (Ig) binding was detected using 125I-labeled anti-mouse Ig (Amersham Biosciences, Piscataway, N.J.).

### Immunocytochemistry of Brain Sections

After euthanasia of control and EcoNDK infected mice, a sagital portion of the cerebral hemisphere was fixed in 10% neutral buffered formalin, then each brain was cut into an average of five coronal sections, dehydrated, embedded in paraffin, and sections of 6μ were cut for staining and microscopy. Sections were subjected to basic indirect immunohistochemical staining using a biotin-avidin system as described (Sharer et al., J. Med. Primatol, 20, 211-217, 1991) with rabbit polyclonal anti-STAT1 sc-346 (Santa Cruz Biotechnology, Santa Cruz, Calif.) binding detected with diaminobenzidine as a chromogen and hematoxylin and eosin counterstaining. Negative control slides were run omitting the primary anti-STAT-1 antibody. Sections were examined in a Zeiss Photomicroscope III and images were captured using a Nikon DN100 digital camera.

### Example 2

Results

### Host Range of EcoHIV in Culture

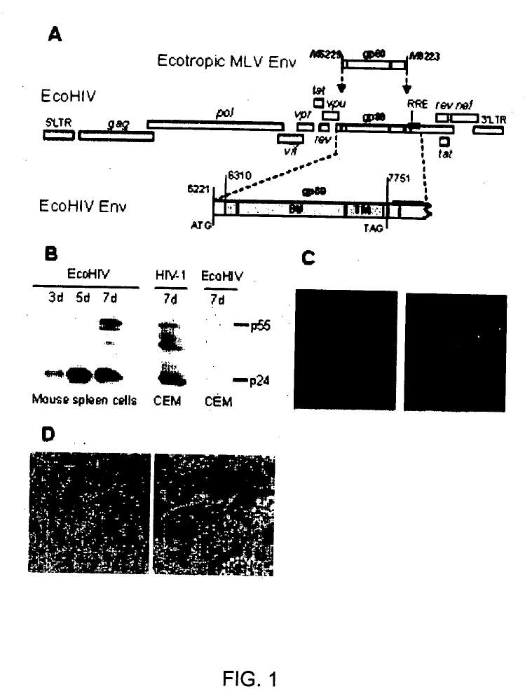

To redirect HIV-1 to infect the rodent, the ecotropic MLV gp80 gene carrying its own stop codon was inserted in-frame into the NL4-3 env gene, preserving the first 90 coding residues, deleting the subsequent 1440, and resuming HIV-1 near the beginning of the gp41 coding region (FIG. 1A). The resulting chimeric virus, EcoHIV, contains all the known coding and regulatory regions of the HIV-1 genome with the exception of gp120; gp41 is unlikely to be expressed because it lacks an in-frame codon for initiation of translation. The biological activity of EcoHIV in culture was tested using several approaches. Mitogen stimulated murine splenic lymphocytes were infected and cells harvested over one week of infection for analysis of the expression of HIV-1 p24 by Western blot (FIG. 1B). Cells of the transformed human T cell line, CEM, were exposed to HIV-1 or to EcoHIV as positive and negative controls, respectively. Fully processed p24 increased in amount with time after infection of mouse cells indicating that it was newly synthesized and properly processed by HIV-1 protease. In contrast, human CEM cells were not susceptible to infection by the EcoHIV. In similar studies the inventors infected primary mitogen stimulated human lymphocytes or transformed mouse cells with EcoHIV and could detect no p24 production. EcoHIV infected mouse lymphocytes were also examined for the presence of viral antigens by indirect immunofluorescence staining with AIDS patient serum or for the presence of syncytia during cocultivation with fresh splenic lymphocytes (FIG. 1 C-D). HIV-1 antigens were detected in EcoHIV infected but not in uninfected mouse lymphocytes, at a frequency of approximately 10%, similar to what was observed during infection of mouse lymphocytes by pseudotyped HIV-1 (Nitkiewicz et al., Journal of Neuro Virology, 10:400-408, 2004), both of which are less efficient infections than the infection of human lymphocytes by HIV-1 in culture. Upon cocultivation EcoHIV infected cells formed large syncytia, indicating that gp80 is properly cleaved to fusion competent proteins. These findings demonstrate that EcoHIV is biologically active, it can productively infect primary murine lymphocytes but not transformed lymphocytes in culture, and that it acquired the host range of ecotropic MLV and is unable to infect human cells.

### Evidence of Persistent Productive Infection of Mice by EcoHIV Including Evidence of Neuroinvasion and Neuropathology and Impaired Immune Function

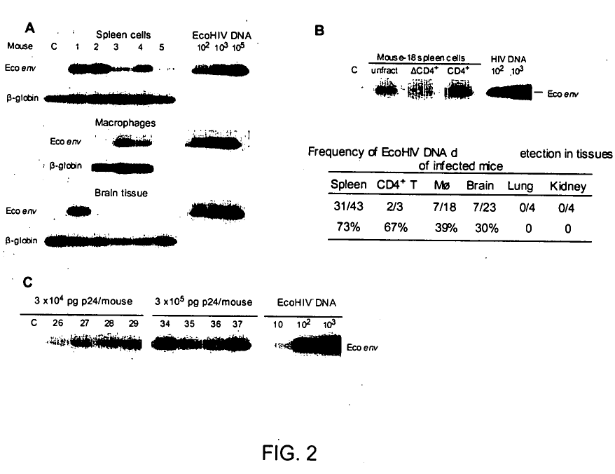

Based upon the demonstration of EcoHIV infection of murine lymphocytes in culture, the inventors tested whether EcoHIV can also establish infection in vivo, in conventional mice (FIG. 2). Adult immunocompetent mice were inoculated by a single intravenous injection of 10⁵ pg p24 EcoHIV. Six weeks after infection or mock-infection, mice were euthanised and tissues were collected for analysis (FIG. 2A). Guided by the cell types infected by HIV-1 in human beings, the inventors tested viral infection in lymphocytes, macrophages, and brain cells by PCR amplification for a region unique to the EcoHIV genome that spans the joint between HIV-1 and MLV. DNA from 5×10⁵ spleen cells or 1.25 mg brain tissue was run in each reaction. Because only about 5-10×10⁵ peritoneal macrophages were obtained from each animal, the entire sample was subjected to amplification. Viral DNA was detected in one or more tissues of 4 of the 5 mice injected. The peak virus burden attained in the spleen at three or six weeks after infection, approximately 1 in 1,000 cells carrying viral DNA, is similar to the range of 1 in 200 to 1 in 20,000 HIV-1 DNA positive cells observed in resting lymphocytes in HIV-1 infected human beings (Chun et al., Proc. Natl. Acad. Sci. USA, 95, 8869-8873, 1998). In contrast to EcoHIV infection in culture, the efficiency of EcoHIV infection in vivo is comparable to that of HIV-1.

The inventors also performed limited investigations of the cell and tissue tropism of EcoHIV in the mouse, testing known infected mice, and the dose response of infection. Splenic lymphocytes were fractionated into CD4-positive and CD4-negative populations prior to DNA PCR for EcoHIV (FIG. 2D). CD4-positive but not CD4-negative lymphocytes carried viral DNA and the DNA burden was higher in CD4-positive cells than in unfractionated cells. In all, 2 out of 3 mice tested carried viral DNA in CD4-positive but not in CD4-negative splenic lymphocytes and none of 4 mice tested carried viral DNA in lungs or kidneys. The lowest dose of EcoHIV tested, 3×10⁴ pg, infected 3 out of 4 mice tested at six weeks after infection and raising the dose 10-fold yielded uniform infection in 4 mice (FIG. 2C). The table in FIG. 2 shows a summary of the results of virus detection in different tissues from six independent experiments of inoculation of mice with EcoHIV at doses from 3×10⁴ pg to 5×10⁵ pg p24 per mouse. EcoHIV was most frequently detected in splenic lymphocytes, but in two mice virus was present in the brain but not the spleen; a total of 33 of 43 mice tested carried EcoHIV DNA in one or more tissues. Overall the initiation of spreading infection after a single exposure, replication competence, infectivity of progeny, and tissue distribution of EcoHIV in the mouse reproduce several important features of the natural infection of human beings by HIV-1.

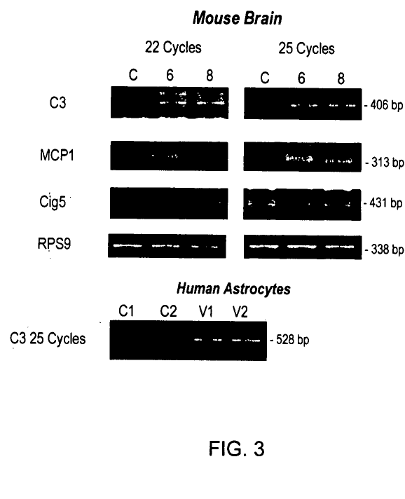

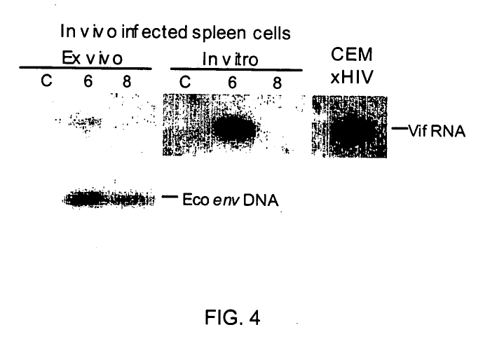

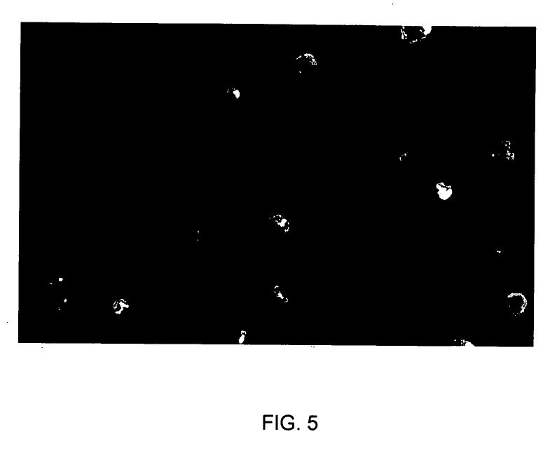

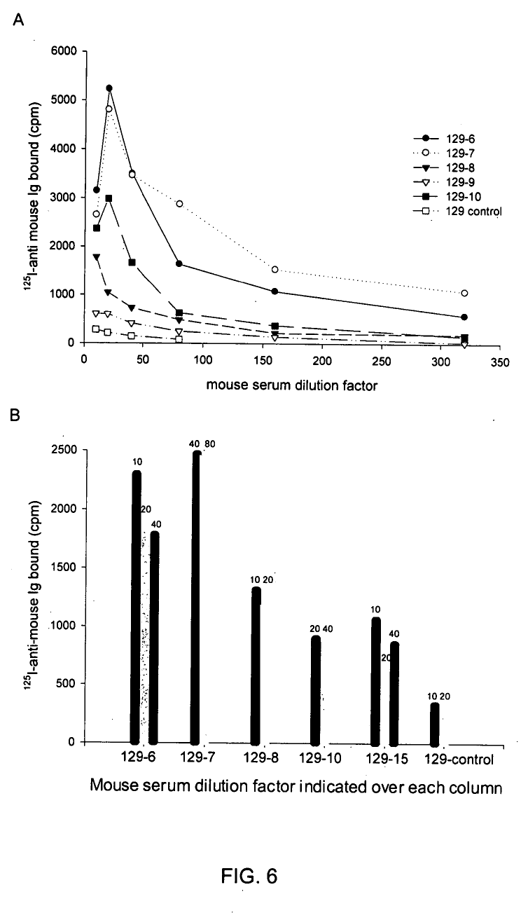

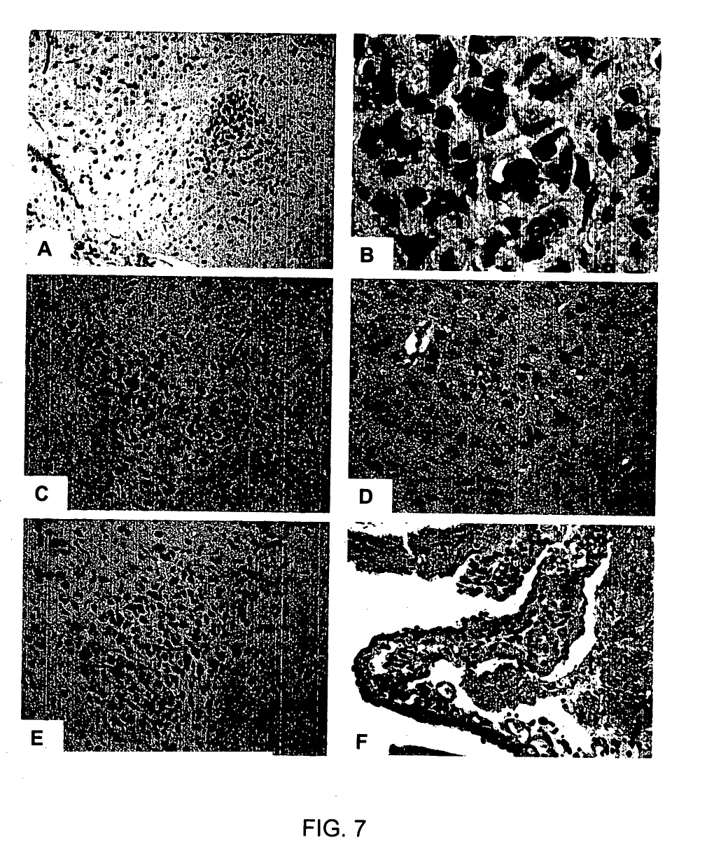

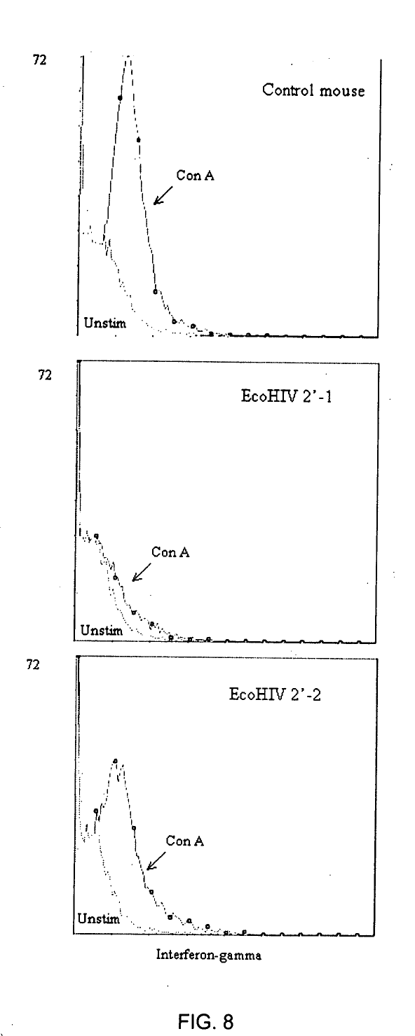

FIG. 2 shows detection of EcoHIV DNA at 6 weeks post inoculation and summarizes findings of several experiments. FIG. 3 shows changes in inflammatory gene expression in infected mouse brains. FIGS. 4 and 5 show evidence of Eco HIV replication in mice. FIG. 6 documents the production and specificity of antiviral antibodies in infected animals, and FIG. 7 shows neuropathological changes at 6 and 12 weeks. FIG. 8 shows impaired immune function in mice receiving two injections of EcoHIV.

As shown in FIG. 2, EcoHIV DNA was found in the spleens of the majority of inoculated mice and at a lower frequency in peritoneal macrophages or in the brain. Infection in the brain may be under-represented because the procedures of DNA extraction and PCR amplification from the brain are currently in the process of being optimized. EcoHIV DNA was present in CD4-bearing lymphocytes and the virus burden in the spleen increased with increasing virus dose. As shown in the table summarizing six experiments, detection of EcoHIV DNA in the spleen was clearly the strongest indicator of infection, and by that measure 73% of EcoHIV inoculated mice became persistently infected with the virus. Thus, viral DNA can be reproducibly detected in a large proportion of EcoHIV infected mice, the virus persists for months after infection, and at least in some animals, virus enters the brain. The presence of EcoHIV DNA in lymphocytes, macrophages, and the brain but not in lung or kidney indicates that EcoHIV has a tissue distribution similar to that of HIV-1 in human beings.

### Brain Pathology

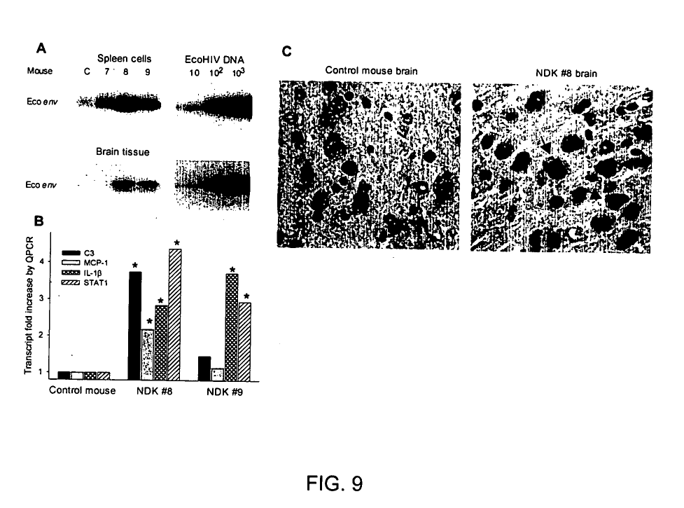

As one measure of neuropathology, brain tissue RNA from these mice was tested for transcriptional activation of genes coding for molecules implicated in neuropathogenesis, such as MCP-1, C3, and the IFN-induced factor Cig5 (FIG. 3). MCP-1 is a marker of HIV-1- and SIV-associated brain disease, elevated C3 was correlated with neuroinflammation, and IFN-related genes are modulated in brains of SIV infected macaques. C3 and Cig5 are modulated specifically in HIV-1 infected human astrocytes in culture, suggesting that they may be useful as cell-specific molecular markers of HIV-1 neuropathogenesis. As shown in FIG. 3, MCP-1 and C3 RNA were significantly increased in brain tissues from infected mice #6 and #8, as was C3 in cultured HIV-1-infected human fetal astrocytes serving as control. Cig5 expression was reduced in infected mice consistent with the temporal pattern of expression of IFN related genes in HIV-1-infected macrophages and astrocytes observed in vitro. FIG. 7 presents direct neuropathological studies in EcoHIV infected mice and FIG. 9 shows changes in host cell gene expression in mice infected by EcoNDK. EcoHIV-infected mouse #8 had brain lesions (FIG. 7) and altered expression of three molecular markers in brain tissue. Mouse #6 had elevated MCP-1 and C3 but no brain lesions, suggesting that pathogenic changes in the brain can potentially be detected in this model by sensitive molecular assays prior to appearance of overt histopathology.

A preliminary histopathological examination of brain tissues from one experiment was performed. Brain lesions containing cellular infiltrates or aggregates were found in the brain of EcoHIV-infected mouse #1 (6 weeks p.i.) and mouse #8 (12 weeks p.i.), but not in brains from control mice or other infected animals. Note a large multinucleated cell in the middle of the lesion in FIG. 7. Similar multinucleated giant cells are the hallmark of HIV-1 encephalitis. Thorough examination of these samples is ongoing, including staining for cell types present in the lesions. These results provide the most direct evidence that EcoHIV infection of mice is pathogenic and in some animals, induces a brain disease highly analogous to that observed in HIV-1 infected patients. EcoNDK is also neuroinvasive and potentially neuropathogenic, see FIG. 9.

### Evidence of HIV-1 Replication In Vivo and Humoral Response to Virus

In seeking direct evidence for virus expression and production in animals, spleen cells isolated from mice infected for 3 months were tested for Vif RNA by RT-PCR either immediately or after stimulation in culture (FIG. 4). Vif RNA is a singly spliced viral transcript produced during HIV-1 replication. Vif RNA signals were detected in directly tested cells from mouse #6 and #9 and culture of mouse #6 cells clearly increased Vif RNA levels indicating virus reactivation or increased virus expression. Consistent with the expression of viral RNA, spleen cells infected in the mouse produced progeny virus that could spread infection in culture (FIG. 5). These results show that EcoHIV is fully replication competent, that it is expressed at low levels in vivo, and that infected cell activation increases virus expression. This pattern of Eco HIV expression in mice is reminiscent of that in HIV-1 infected patients.

In natural virus infections, antiviral immune responses generally accompany ongoing virus replication. Because immunocompetent mice were used for EcoHIV inoculation, detection of antiviral responses is an indicator of the endogenous production of viral proteins through active EcoHIV infection as well as direct evaluation of the ability of the mouse to mount anti-HIV immune responses to HIV-1 antigens presented during infection. Using sera from mice 3 months after infection the humoral response to HIV-1 structural protein Gag or regulatory protein Tat was tested by radioimmunoassay (FIG. 6). Serum from mouse #15 was collected six weeks after infection. At 12 weeks after infection, four out of five mice also had antibodies to HIV-1 Gag and Tat (FIG. 6) and also carried viral DNA in the spleen; mouse #15 was both seropositive and positive for viral DNA six weeks after infection. Mouse #9 carried no viral DNA in the spleen and was seronegative for antiviral antibodies. Initial studies indicated that the titers of the mouse antibodies ranged from 1:40 to >1:320. The presence of an antiviral immune response at 3 months after infection and induction of a response against viral Tat which is not present in the virus particle indicate continuing virus replication in the body as well as the fact that the humoral immune response to virus in mice remains functional. Both observations are important for the feasibility of testing vaccine using the EcoHIV model of mouse infection. The concordance between detection of viral DNA and detection of antiviral antibodies provides another confirmation of active virus replication and spread in EcoHIV infected mice.

### Preliminary Findings of Impaired Immune Function After EcoHIV Infection

FIG. 8 provides preliminary data that EcoHIV can induce impairment of immune function in infected mice. In a pilot study, lymphocytes from mice receiving two injections of EcoHIV failed to respond to immune activation in culture by the production of the key cytokine interferon-gamma. These findings raise the possibility that further modification of the EcoHIV infection protocol may result in immune deficiency in mice.

### Infection of Mice by a Virus Chimera Based Upon an African HIV-1

To determine the general applicability of this approach to the study of different natural HIV-1 species in mice, the inventors constructed EcoNDK with the MLV gp80 gene inserted into NDK, a highly cytopathic Clade D HIV-1 (Ellrodt et al., Lancet, 1, 1383-1385, 1984). EcoNDK contains the gag gene of NDK that contributes to its high virulence in culture (de Mareuil et al., J. Virol., 66, 6797-6801, 1992) and might enhance its activity in the mouse. Three weeks after inoculation with 10⁵ pg p24 EcoNDK three mice were euthanised and spleen and brain were collected. Viral DNA was detected in splenic lymphocytes from each mouse, and two of the three mice also carried viral DNA in the brain (FIG. 4A). NDK8 had about 1 copy of viral DNA per 5,000 lymphocytes, comparable to mice infected by EcoHIV (FIG. 2) and also comparable to HIV-1 infected persons (Chun et al., Proc. Natl. Acad. Sci. USA, 95, 8869-8873, 1998). In addition, this is the earliest time that the inventors assayed mouse brain tissue for the presence of chimeric HIV-1 and it is clear that the virus can invade the brain by three weeks after exposure.

Immune deficiency or neurological impairment by HIV-1 infection of human beings takes years to develop and indeed the inventors have observed no overt signs of immune dysfunction or marked encephalitis in EcoHIV infected mice, most of which were euthanised six weeks after inoculation. However, molecular markers of cellular abnormalities associated with SIV infection or HIV-1 infection in the brain have been described, some of which predict later neurological disease (Zink et al., J. Infect. Dis., 184, 1015-1021, 2001; Conant et al., Proc. Natl. Acad. Sci. USA, 95, 3117-3121, 1998; Lane et al., Mol. Med., 2, 27-37, 1996; Roberts et al., Am. J. Pathol., 162, 2041-2057, 2003). To investigate subtle changes that may occur early in HIV-1 infection of the mouse, the inventors determined the expression of complement component C3, IL-1β, IL-6, MCP-1, and STAT-1 that are among the factors that influence inflammatory or antiviral responses to HIV-1 in the brain. QPCR was conducted, expression normalized to a housekeeping transcript, and the data are reported as fold increase relative to transcript levels in brain tissue of the control mouse (FIG. 4B). NDK 8, which had the highest virus burden in spleen and the brain, showed significant increases in the expression of C3, IL-1β, MCP-1, and STAT-1. Increased expression of C3 was also seen in viral DNA positive brains from two mice infected by EcoHIV (not shown). NDK 9 had lower levels of EcoNDK in brain and spleen and showed significant increases in the expression of IL-1β and STAT-1, but not in C3 and MCP-1. IL-6 expression was similar in NDK 8, NDK 9, and the control mouse brain (not shown). Because STAT-1 in NDK 8 was the most highly induced transcript observed, the inventors tested STAT-1 protein expression in cortical brain sections of NDK 8 versus the control mouse (FIG. 4C). Increased expression of STAT-1 protein was observed in cytoplasm of neurons in NDK 8 relative to the control but not in other cell types. These findings indicate that the approach to construction of HIV-1 tropic to mice can be generalized to different HIV-1 backbones. Moreover they indicate that in only a few weeks of infection, chimeric HIV-1 elicits cellular responses in mouse brain like those seen in HIV-1 or SIV infection in the brain (Zink et al., J. Infect. Dis., 184, 1015-1021, 2001; Conant et al., Proc. Natl. Acad. Sci. USA, 95, 3117-3121, 1998; Lane et al., Mol. Med., 2, 27-37, 1996; Roberts et al., Am. J. Pathol., 162, 2041-2057, 2003).

As shown in FIG. 9, EcoNDK, a chimeric virus based upon an African Clade D HIV-1 and MLV is also replication competent. Three weeks after inoculation, all mice tested carried EcoNDK DNA in the spleen. Two mice also carried viral DNA in the brain and these mice suffered changes in cellular RNA expression in the brain consistent with the presence of the virus. It is noteworthy that the cellular genes activated by exposure to EcoNDK have also been found to be activated by exposure of human cells to HIV-1. In addition, STAT-1 expression in neurons, as shown in the brain of EcoNDK infected mouse #8, has also been observed in macaques suffering SIV encephalitis. These findings indicate the generality of the approach of constructing HIV-1 competent to replicate in mice and alter host physiology. Moreover, it shows that the approach can be used to investigate the replication and control of HIV-1 of African origin.

### Insertion of gp120 Domains into gp80 of EcoHIV

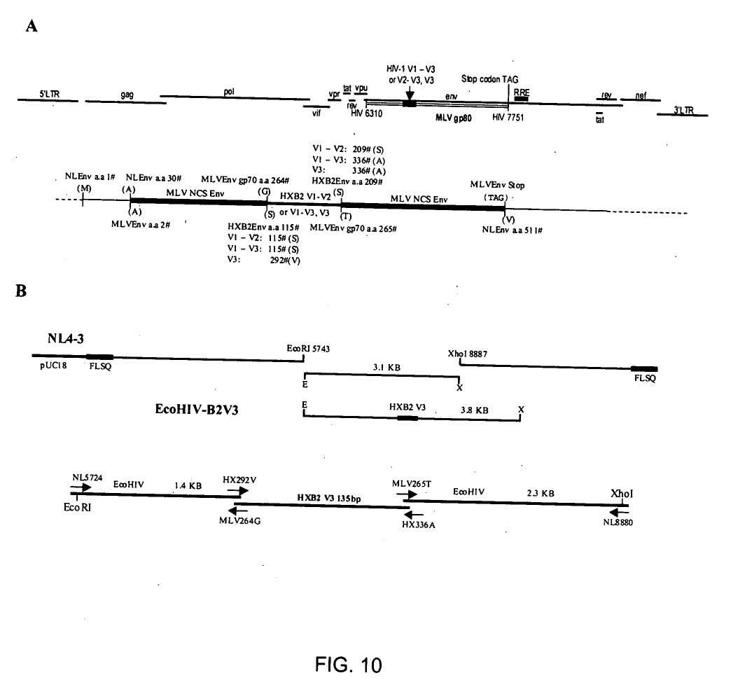

Because the binding of the HIV-1 envelope glycoprotein to human cells has often been proposed as pathogenic a strategy was developed for reconstructing EcoHIV to include gp120 regions V3, V1/V2, or V1-V3 implicated in binding certain cellular coreceptors. The summary of steps for these insertions from HXB-2 into gp80 in EcoHIV is shown in FIG. 10. The insertion point is between amino acid 264 and 265, within the proline-rich region that tolerated insertion of GFP and yielded viable MLV-GFP virus. Several viral clones were obtained, V3 insertion was confirmed by sequencing. Transfection DNA into human embryonic kidney cells yielded viable virus in each case indicating that these insertions can be tolerated. The option for insertion of potentially pathogenic or antigenic domains from HIV-1 gp120 into functional EcoHIV may be valuable for reproducing certain aspects of HIV-1 mediated pathogenesis in the mouse model or for induction of anti-gp 120 immune responses in vaccine development.

### Discussion

The mouse model of HIV-1 infection introduced here consists of inoculation of conventional mice with a chimeric HIV-1 that employs species-specific cellular receptors to enter mouse cells. The infection spreads to multiple organs, induces antiviral immune responses, and alters cellular gene expression in the brain. Because the ecotropic envelope that they carry does not mediate entry into human cells, EcoHIV and EcoNDK are less hazardous than are HIV-1 and SIV. These chimeric viruses will be useful for modeling many aspects of HIV-1 infection of human beings in a tractable animal host.

The infectivity of EcoHIV in murine lymphocytes in culture demonstrates that the HIV-1 genome tolerates the insertion of the MLV envelope-coding region and that the essential cis-regulatory elements of HIV-1 as well as all the coding regions are functional. It also indicates that the ecotropic gp80 associates with the HIV-1 core to mediate virus entry, as has been shown in studies analogous to ours in construction of a replication competent chimera of HTLV-1 and MLV (Delebecque et al., J. Virol., 76, 7883-7889, 2002). Because HIV-1 protease is active at the cell membrane during virion budding (Kaplan et al., Journal of Virology, 68, 6782-6786, 1994) and can cleave MLV p15 to p12 (Kiernan et al., Journal of Virology, 72, 9621-9627, 1998), fusogenic p12 is likely to be present at the cell surface to mediate the observed cell fusion that is not generally seen with MLV. This gain of function may facilitate cell-to-cell transmission of the virus in the mouse. In culture, the host range of EcoHIV reproduces that of ecotropic MLV, EcoHIV infects mouse but not human lymphocytes, but only a minority of cells are infected, raising the possibility that EcoHIV targets a sub-population of cells. This proposal is under investigation. In the mouse, ecotropic MLV replicates in T lymphocytes with both envelope and the viral LTR contributing to tropism (Rosen et al., Journal of Virology, 55, 862-866, 1985; Evans et al., J. Virol., 61, 1350-1357, 1987) but EcoHIV replicates in macrophages and the brain, as well as in lymphocytes. Neurotropism and neuropathogenesis are common features of lentiviruses including HIV-1, in part because of their replication in macrophages (Patrick et al., J. Virol., 76, 7923-7931, 2002). However, HIV-1 gp120 that targets the virus to human macrophages as well as T cells is absent from EcoHIV. The LTR present in EcoHIV does not influence HIV-1 tropism in cell culture (Pomerantz et al., Journal of Virology, 65, 1041-1045, 1991) but it is possible that it affects the EcoHIV host range the inventors observed in the animal. Further research is required to determine the basis for viral infection and expression in different tissues in EcoHIV-infected animals.

One inoculation was sufficient to establish EcoHIV infection in more than 75% of the mice tested and viral DNA was detected in the major target cell types of HIV-1. EcoHIV and EcoNDK reached virus burdens in the spleen comparable to HIV-1 burdens in resting lymphocytes in human beings (Chun et al., Proc. Natl. Acad. Sci. USA, 95, 8869-8873, 1998), indicating that both viruses are significantly infectious under the conditions employed. The extent of infection by EcoHIV was somewhat lower at twelve weeks after infection than at three or six weeks (not shown). This could arise from a self-limiting infection or from effective antiviral immunity. The presence of EcoHIV in multiple organs and its transmission from spleen cells in culture indicate that the progeny virus is highly infectious. On the other hand, EcoHIV infected mice consistently produced anti-Gag and anti-Tat antibodies. Therefore the inventors favor the second possibility: that infected mice mount immune responses that control EcoHIV infection, at least temporarily. This host-virus balance is reminiscent of the first years of HIV-1 infection in human beings while the immune system is intact and the infection is controlled (Pantaleo et al., Nature Medicine, 10, 806-810, 2004). However, in the absence of therapy the balance later shifts to increased viral replication, loss of immune function, and development of disease in the immune and nervous systems. The inventors are currently investigating the consequences of long-term infection of mice by EcoHIV and EcoNDK to determine whether a similar pathogenic shift takes place.

Although no overt disease was been detected within six weeks of infection, EcoNDK activated IL-1β, MCP-1, and STAT-1 expression, cellular responses in the brain associated with later disease in HIV-1 or SIV infection. IL-1β over-expression has been observed in the brain during SIV-induced neurological disease (Lane et al., Mol. Med., 2, 27-37, 1996) and can be induced in the brain by viral Tat itself (Philippon et al., Virology, 205, 519-529, 1994). STAT-1 was induced in astrocytes, microglia, and neurons during SIV encephalitis (Roberts et al., Am. J. Pathol., 162, 2041-2057, 2003), its induction was confined to neurons in the brain of EcoNDK infected mice tested three weeks after inoculation. MCP-1 can be induced in the brain by viral Tat alone (Pu et al., Mol. Cell Neurosci., 24, 224-237, 2003), it has been detected in the brains of human beings suffering from HIV-1 associated dementia (Conant et al., Proc. Natl. Acad. Sci. USA, 95, 3117-3121, 1998) and indeed induction of MCP-1 in the central nervous system has been described as a predictor of later brain disease in an SIV model of encephalitis (Zink et al., J. Infect. Dis., 184, 1015-1021, 2001). EcoNDK infection of mice thus mimics human or monkey infection by primate lentiviruses in activation of cellular gene expression in the brain, possibly as a consequence of expression of HIV-1 Tat.

EcoHIV infection of mice reproduces key characteristics of HIV-1 infection of human beings including host cell range, early neuroinvasiveness, systemic immune responses, and induction of inflammatory and antiviral responses in the brain. Further modification of the EcoHIV construct to increase virulence may tip the balance observed during the early weeks of mouse infection from immune response to immune dysfunction. However, it should be clear that the control of EcoHIV infection in the context of antiviral immune responses is an excellent starting point for vaccine studies. The inventors believe that EcoHIV provides a link between the extensive range of experimental models established in mice and the knowledge base of HIV-1 molecular genetics to investigate HIV-1 infection in a tractable animal host.

All publications referenced herein are hereby incorporated in their entirety. While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A chimeric HIV-1 construct capable of infecting a rodent cell, comprising coding and regulatory regions of the HIV-1 genome, and a heterologous viral envelope, wherein the coding and regulatory regions of the HIV-1 genome are derived from a molecular clone of any clade or construction.

2. The chimeric construct of claim 1, wherein the complete or partial coding region of gp120 is replaced by the coding region for a heterologous viral envelope.

3. The chimeric viral construct of claim 2, wherein the complete or partial coding region of gp120 is replaced by the coding region for ecotropic murine leukemia virus gp80.

4. A propagation competent rodent model of HIV-1 in which at least the somatic cells are susceptible to the construct of claim 1, wherein expression of the construct is sufficient to effect phenotypic changes consistent with HIV-1 pathology.

5. The model of claim 4, wherein the animal is a mouse.

6. The model of claim 4, wherein the animal is a rat.

7. A virus propagation competent rodent model of AIDS in which at least the somatic cells are susceptible to the construct of claim 1, wherein expression of the construct is sufficient to effect phenotypic changes consistent with AIDS pathology.

8. The model of claim 7, wherein the animal is a mouse.

9. The model of claim 7, wherein the animal is a rat.

10. A method for producing a rodent model of HIV infection comprising administering the construct of claim 1 to a rodent.

11. A method for producing a rodent model of AIDS comprising administering the construct of claim 1 to a rodent.

12. A virus propagation competent rodent model of HIV-1 in which at least the somatic cells are susceptible to infection by the construct of claim 1, wherein expression of the construct is sufficient to effect phenotypic changes consistent with HIV-1 pathology, and wherein the model is suitable for testing the efficacy of HIV-1 directed immunogenic constructs or vaccines for prevention of infection in a subject inoculated with the construct of claim 1.

13. A virus propagation competent rodent model of HIV-1 in which at least the somatic cells are susceptible to infection by the construct of claim 1, wherein expression of the construct is sufficient to effect phenotypic changes consistent with HIV-1 pathology, and wherein the model is suitable for testing the efficacy of HIV-1 directed immunogenic constructs or vaccines for amelioration of disease in a subject inoculated with the construct of claim 1.

14. A virus propagation competent rodent model of HIV-1 in which at least the somatic cells are susceptible to infection by the construct of claim 1, wherein expression of the construct is sufficient to effect phenotypic changes consistent with HIV-1 pathology, and wherein the model is suitable for testing the efficacy of a pharmaceutically or veterinarilly suitable composition for amelioration of disease in a subject inoculated with the construct of claim 1.


SEQUENCE LISTING (3 sequences)

SEQ ID NO: 1 - DNA, 27 bp, Artificial sequence
  PCR primer
atgatctgtagtgctgcgcgttcaacg

SEQ ID NO: 2 - DNA, 28 bp, Artificial sequence
  PCR primer
gagccgggcgaagcagtactgacccctc

SEQ ID NO: 3 - DNA, 28 bp, Artificial sequence
  DNA probe
ggttaacccgcgaggccccctaatcccc